(19) United States
(12) Patent Application Publication
(10) Pub. No.: US 2016/0331927 A1
(43) Pub. Date: Nov. 17, 2016

(54) Ventricular Catheter

(71) Applicant: THE REGENTS OF THE UNIVERSITY OF COLORADO, A BODY CORPORATE, Denver, CO (US)

(72) Inventors: Dusty Richardson, Denver, CO (US); Ken Winston, Golden, CO (US)

(73) Assignee: The Regents of the University of Colorado, a body corporate, Denver, CO (US)

(21) Appl. No.: 15/112,396

(22) PCT Filed: Jan. 30, 2015
(86) PCT No.: PCT/US15/13710

§ 371 Date: Jul. 18, 2016

Related U.S. Application Data
(60) Provisional application No. 61/934,567, filed on Jan. 31, 2014.

(51) Int. Cl. A61M 25/00; A61M 25/01; A61M 27/00

(52) U.S. Cl. CPC A61M 25/007; A61M 27/006; A61M 25/0102; A61M 2025/0063; A61M 2202/0464; A61M 2210/0693

(57) ABSTRACT

The present invention provides a ventricular catheter designed to prevent or reduce CSF shunt obstruction. In particular, the ventricular catheter of the invention comprises a tube having a distal end and a tapered proximal end, in which the tapered proximal end comprises a plurality of slotted openings.

### FIELD OF THE INVENTION

The present invention relates to a ventricular catheter designed to reduce cerebrospinal fluid (CSF) shunt obstruction. In particular, the ventricular catheter of the invention comprises a tube having a distal end and a tapered proximal end, in which the tapered proximal end comprises a plurality of slotted openings.

### BACKGROUND OF THE INVENTION

Ventricular cerebrospinal fluid (CSF) shunting procedures are one of the most common procedures performed by neurosurgeons with approximately 25,000 procedures done in the U.S. every year. The procedure involves placing a small tube into the fluid-filled space of the brain (ventricle) and diverting the fluid through a valve mechanism and into another place in the body where it can be absorbed (such as the abdomen or large vein). The catheter that is placed into the brain is often referred to as the “proximal shunt catheter” or “ventricular catheter.” An example of conventional ventricular catheter is VentriClear EVD Catheter available from Medtronic (Minneapolis, Minn.).

Occlusion of the proximal shunt catheter is the most common causes of shunt failure (Pediatr Neurosurg. 2000; 33(5), 230-236) requiring additional surgical procedures to correct. Proximal shunt catheter occlusion typically occurs from blockage of the draining holes caused most commonly by either ingrowth of choroid plexus or blockage of the holes from fragments of the lining of the ventricles. Revisions of the ventricular catheter are often difficult as a result of the ingrowth of the choroid plexus into the holes of the catheter which causes the catheter to become fixed within the brain. Significant bleeding into the brain is a not-infrequent problem when trying to remove catheters that are fixed in position from ingrowth of choroid plexus.

Accordingly, there is a need for a new ventricular catheter that can significantly reduce or prevent occlusion, e.g., obstruction from tissue invasion.

### SUMMARY OF THE INVENTION

Some aspects of the invention provide a proximal shunt catheter that is less prone to obstruction and is significantly easier and safer to replace, if and when necessary. The ventricular catheter of the invention significantly reduces the likelihood of tethering, adhesion or ingrowth of the choroid plexus to the ventricular catheter. In fact, it is believed that the design of the ventricular catheter of the invention prevents tethering, adhesion or ingrowth of the choroid plexus due to its unique design. In particular, the ventricular catheter comprises a plurality of slotted openings (or elongated apertures) without a closed terminus. Additionally, the ballistic tapered design of the catheter tip as well as each of fingers/slots is designed to allow for safer removal. The ventricular catheters of the invention can be easily placed using currently-employed techniques known to one skilled in the art (i.e., planted within a patient's brain) and is highly cost-effective to produce.

A ventricular catheter of the invention comprises a tube. The tube comprises a distal end; and a tapered proximal end. The ventricular catheters of the invention are configured to be placed within a patient's brain, as such the catheters are made of materials comprised of a biocompatible material. At minimum, the outer surface of the catheters is comprised of biocompatible material, but typically the entire catheter is made of biocompatible material. The term “biocompatible” refers to a material that is not toxic to cells. In some embodiments, a substance is considered to be “biocompatible” if the presence of material in vivo results in approximately ≦20%, typically ≦10%, often ≦5% and most often ≦1% cell death. In some embodiments, a substance is considered to be “biocompatible” if its presence in vivo does not induce inflammation and/or other adverse effects in vivo. The terms “approximately” and/or “about” as used herein when referring to a numeric value means ±20%, typically ±10%, and often ±5% of the numeric value.

The tapered proximal end of the ventricular catheter comprises a plurality of slotted openings or slits. The term “slotted opening” and/or “slit” refer to an elongated aperture or opening that is present from the beginning of the orifice of the tapered proximal end and traversing towards the distal end. It should be appreciated that the slotted opening or slit need not be straight or even linear. It can be curved, wavy, etc. Typically, the proximal end of the ventricular catheter comprises two, three, four or more, often two, three or four, more often two, three or four, and most often four slotted openings or slits. The size and the length of each slotted openings or slits can vary depending on, for example, the patient's size and age. In some embodiments, the interior surface of the tapered proximal end of the tube comprises a protrusion or protuberance. Typically, this protrusion is located at or near the start of the tapered proximal end. The protrusion can form a ring within the interior surface or it can be of one of more, typically two or more, often three or more, and more often four or more protuberances. The protrusion can serve a wide variety of functions. One of its functions is as a stopper for a stylet as described in more detail below.

In some embodiments, the proximal end of the ventricular catheter comprises at least three, typically four or more, and often four slotted openings. The slotted openings of the ventricular catheter can run substantially (i.e., at least 80%, typically at least 90%, and often at least 95% of) the entire length of the tapered proximal end. The term “tapered proximal end” or “tapered end” refers to a portion of the ventricular catheter in which the diameter of the tube gradually decreases. The angle of decline can vary depending on a variety of factors such as, but not limited to, the length of the catheter, the length of the tip of the catheter, the area of implantation, etc. However, it should be appreciated that in general, the diameter at the tip of the catheter is smaller than the diameter of the catheter body.

Typically, the length of the tapered proximal end is at least 25 mm, often at least 20 mm, and more often at least 10 mm.

Other aspects of the invention provide a device comprising a ventricular catheter as disclosed herein, and a stylet. The stylet is capable of being removable fitted within the ventricular catheter. In addition, the stylet comprises a distal end, and a beveled proximal end. The beveled proximal end of the stylet is capable of interacting with the tapered proximal end of the ventricular catheter, thereby limiting the length of the stylet that can be inserted into the ventricular catheter.

In some embodiments, the interior surface of the tapered proximal end of the ventricular catheter comprises a protrusion as described herein to limit the length of the stylet inserted into the ventricular catheter.

Yet in other embodiments, the beveled proximal end of the stylet interacts with the protrusion to limit the length of the stylet inserted into the ventricular catheter.

The terminal end of the catheter (the terminal intraventricular portion or the proximal end) is open and is in continuity with the slots. As discussed above, the slots are typically continuous with the opening at the proximal end of the catheter without any intervening material that separates the slots from the orifice of the proximal end.

Another aspect of the invention provides a method for reducing the incident of proximal shunt catheter occlusion in a subject who is in need of a proximal shunt catheter. Such a method includes using a proximal shunt catheter of the invention. In general, use of the proximal shunt catheter of the invention reduces the incident of catheter occlusion by at least about 10%, typically at least about 20%, and often by at least about 40% compared to the catheter occlusion occurring using conventional ventricular catheter after one, two, three or five years. Alternatively, using the ventricular catheter of the invention results in about 25% or less, typically about 30% or less, often about 40% or less, and more often about 50% or less occlusion after one, two, three or five years compared to a subject who is fitted with a conventional ventricular catheter. The term “about” refers to ±20%, typically ±10%, and often ±5% of the numeric value. It should be appreciated that improvements in reducing the amount and/or the incident of occlusion are statistically derived values at p-value ≦0.1, typically p-value ≦0.05, and often p-value ≦0.01.

Another aspect of the invention provides a method for reducing the incident and/or the amount of ingrowth of choroid plexus in a ventricular shunt catheter, said method comprising placing a proximal shunt catheter of the present invention to a subject in need of a ventricular catheter. The use of proximal shunt catheter of the invention reduces the amount and/or incident of ingrowth of choroid plexus by at least about 10%, typically by at least 25%, often by at least 40%, and more often by at least 50% compared to using a conventional ventricular shunt catheter, after one, two, three, or five years.

### BRIEF DESCRIPTION OF THE DRAWINGS

FIG. 1 is a schematic illustration of a typical conventional ventricular catheter;

FIG. 2 is a cross-sectional schematic illustration of one embodiment of the ventricular catheter of the present invention;

FIG. 3 is a cross-sectional schematic illustration of another embodiment of the ventricular catheter of the present invention;

FIG. 4 is a front-end view schematic illustration of the proximal end of one embodiment of the ventricular catheter of the present invention;

FIG. 5 is a schematic illustration of one particular stylet that can be used with the ventricular catheter of the present invention; and

FIG. 6 is a schematic illustration of one embodiment of the cross-sectional view of the ventricular catheter of the invention with a stylet inserted therein.

FIG. 7 is a graph of comparative flow rate vs. volume of a catheter of the invention vs. a conventional catheter.

FIG. 8 is a graph showing a comparative flow volume at various pressures between the catheter of the invention and a conventional catheter.

FIGS. 9A and 9B are pictures of some of the representative embodiments of the invention.

FIGS. 10A and 10B are line drawings of FIGS. 9A and 9B, respectively.

### DETAILED DESCRIPTION OF THE INVENTION

The present invention will be described with regard to the accompanying drawings which assist in illustrating various features of the invention. In this regard, the present invention generally relates to a ventricular catheter. That is, the invention relates to a ventricular catheter that prevents or significantly reduces likelihood of occlusion. It should be appreciated that the accompanying drawings are provided solely for the purpose of illustrating the practice of the present invention and do not constitute limitations on the scope thereof.

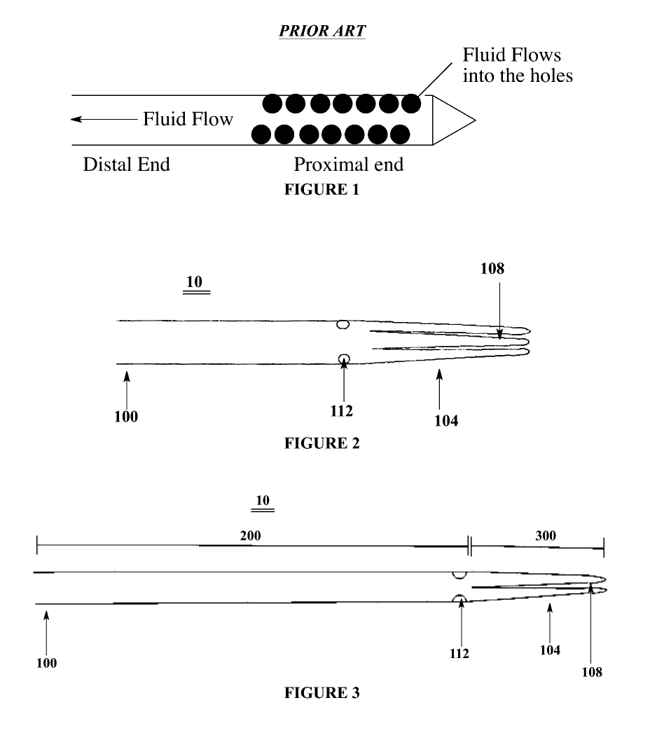

FIG. 1 shows a conventional ventricular catheter that has a plurality of holes on the distal end. Typically, the distal end of the catheter is attached to a valve which controls CSF flow rate. Fluid flows into the small holes and drains out at the distal end of the catheter. Because the catheter is implanted in the ventricle through the brain, it is common for these small fenestrations to become occluded with tissue (e.g., choroid plexus, blood cells, tumor cells, suctioned ependymal tissue) or proteins. Obstruction of the ventricular catheter often results in the need for emergency neurosurgical operation to remove or replace the catheter.

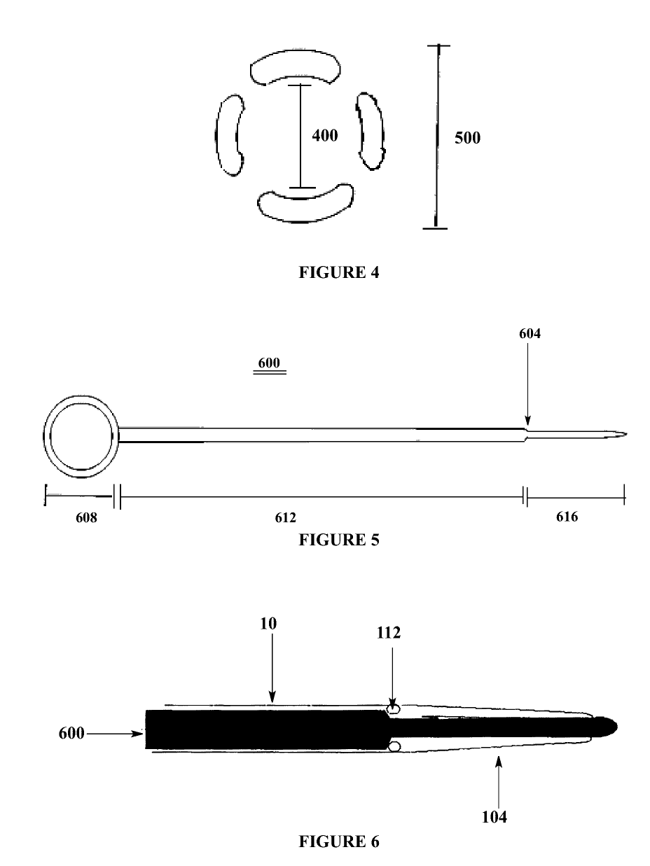

As illustrated in FIGS. 2-6, ventricular catheter 10 of the present invention comprises a distal end 100 and a tapered proximal end 104. The tapered proximal end 104 includes a slotted opening or a slit 108 which allows fluid (e.g., CSF) to flow into the ventricular catheter 10 and drains out at the distal end 100. The number of slotted openings or slits can be two, three, four, or more. The size and the length of slotted openings or slits can vary depending on a variety of factors including the size and the age of the patient as well as the rate of fluid flow desired. Typically, the length of the slotted opening or the slit is from about 1 mm to about 20 mm, often from about 2 mm to about 10 mm, and most often from about 2 mm to about 5 mm. The largest gap in the slotted opening or the slit is typically from about 0.1 mm to about 3 mm, often from about 0.1 mm to about 2, and most often from about 0.5 mm to about 1 mm. However, it should be appreciated that the scope of the invention is not limited to these lengths and gaps. In some embodiments, the tip of the proximal end 104 comprises fluted edges.

The proximal end 104 is tapered from the base to the tip. This tapered design allows easy withdrawal or removal from the ventricle, if and when necessary. The proximal end 104 typically tapers in a conical fashion. Such a configuration allows close approximation with the stylet 600.

The ventricular catheter of the invention is typically made with the same flexible material of the conventional ventricular catheters. The tapered proximal end 104 is often made from a semi-rigid materials commonly used in CSF-draining devices.

The ventricular catheter of the present invention typically has no holes, which are used in conventional ventricular catheters to allow fluid flow. Absence of holes makes the ventricular catheter of the present invention less prone to obstruction by cells, proteins or other ventricular debris. The ventricular catheter of the present invention can be inserted into the brain by the same methods as currently used catheters. Such methods are well known to one skilled in the art.

The total length (e.g., the sum of lengths 200+300) of ventricular catheter can vary and one skilled in the art can readily determine the minimum length required to allow removal of CSF from ventricle to a desired area. Generally, the length of 200 is about 15 cm, typically about 12.5 cm, often about 10 cm. The length of the proximal end 104 is about 2.5 cm, typically about 2 cm, often about 1.5 cm, and more often at least about 1 cm. It should be appreciated, however, that the scope of the present invention is not limited to such lengths. In fact, the overall length as well as the length of the proximal end 104 can be longer or shorter depending on a variety of factors such as the age and the size of the patient, the rate and/or the volume of CSF to be drained, etc.

The outer diameter 500 of the tip of proximal end 104 is generally about 5 mm or less, typically about 4 mm or less, often about 3 mm or less, and more often about 2 mm or less. The inner diameter 400 of the tip of proximal end 104 is generally about 2 mm or less, typically about 1.5 mm or less, and often about 1 mm or less than the outer diameter 500. However, it should be appreciated that the scope of the invention is not limited to these specific diameter configurations. Both the inner and the out diameters of the tip of proximal end 104 can vary significantly depending on a variety of factors such as those described above as well as the material of the ventricular catheter.

The ventricular catheter 10 can also include a protrusion 112 within the interior surface. The protrusion 112 can form a complete circle (i.e., ring) within the interior surface of the ventricular catheter. The protrusion is typically located at or near the start of the tapered proximal end 104. The protrusion can also be one or more protuberances that is present within the interior of the ventricular catheter.

The protrusion 112 limits the length of the stylet 600 that can be inserted into the ventricular catheter 10. The stylet 600 has a beveled portion 616 which catches or is stopped by the protrusion 112. Typically, the protrusion 112 comprises a rigid material (e.g., rigid plastic ring) on the interior of the often flexible ventricular catheter 10. In some embodiments, the stylet 600 has dovetailed ridges which fit between the fingers that are formed from the slotted openings 108 of the ventricular catheter. Such configurations of the stylet 600 and ventricular catheter 10 allow smooth insertion of ventricular catheter 10 and easy withdrawal of stylet 600 after placement of ventricular catheter 10 within a patient.

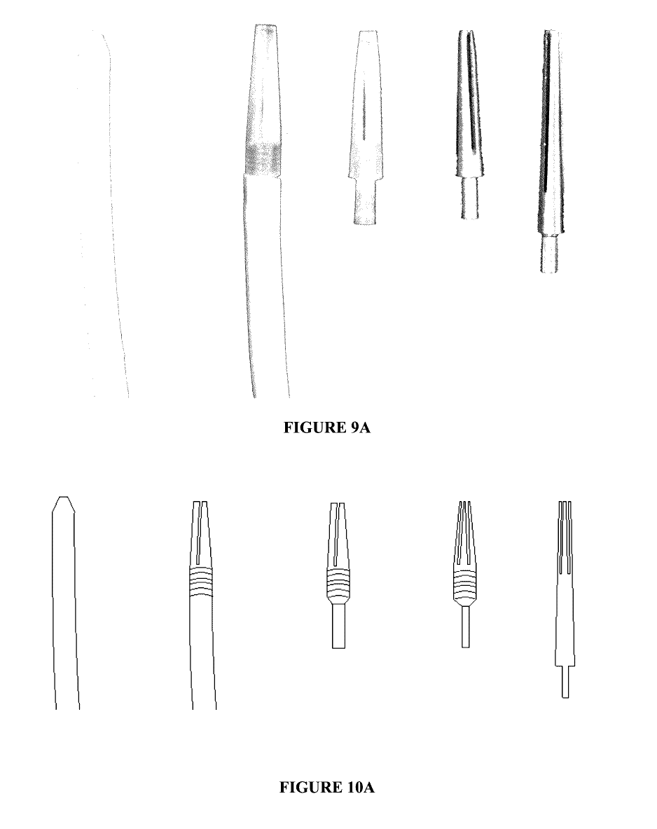

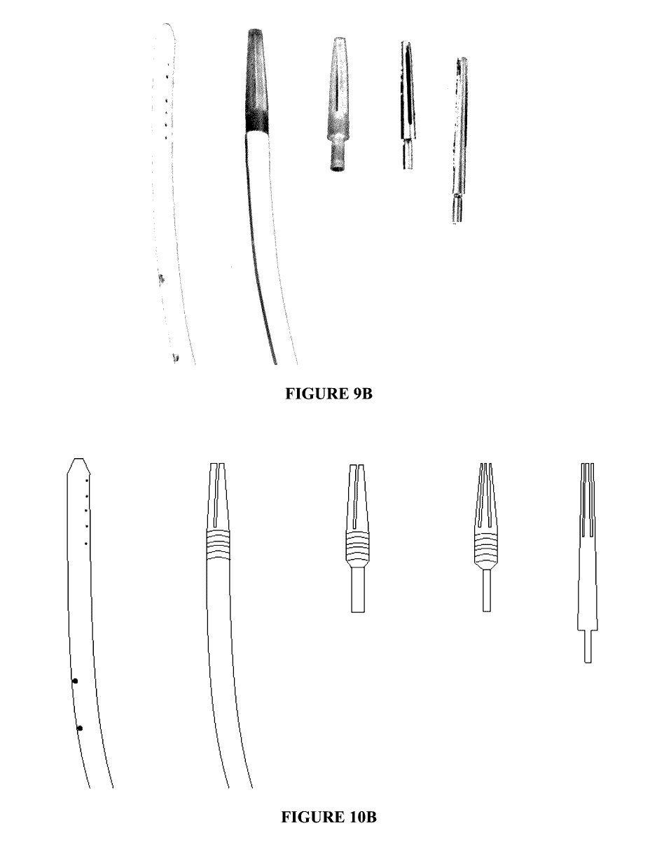

FIGS. 9A and 9B show some of the other embodiments of the invention. FIGS. 10A and 10B are line drawings of FIGS. 9A and 9B, respectively. As can be seen in FIGS. 9A-10B, in some embodiments, the ventricular catheter can comprise a separate tip-end. In particular, as can be seen the tubing and the tip-end can be threaded together to form a single unit. The tip-end unit corresponds to the proximal end comprising a plurality of slotted openings or slits as described herein. The non-tapered portion of the tip-end can be threaded or it can be simply inserted into the tubing. In one particular embodiment, the inner surface of the tubing comprises a complementary thread so that the tip-end is threaded into the inner diameter of the tubing to affix the tip-end into the tubing. When no threading is used, the outer diameter of the tip-end and the inner diameter of the tubing are configured such that when the narrower portion of the tip-end is inserted into the tubing, they form a snug fit to prevent accidental removal of the tip-end from the tubing. In general, the largest outer diameter of the end unit will correspond to the outer diameter of the tubing.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

The catheter of the invention (sometimes referred to herein as “Winston-Richardson catheter”) is inter alia designed to promote less proximal shunt catheter obstruction and ease as well as safety of removal upon revision surgery. In designing the catheter, care was taken to not compromise the rate of flow through the catheter at standard intracranial pressures (0-26 cm H₂O).

In order to demonstrate equivalency of flow, the catheter of the invention was tested simultaneously along side a commercially-available (i.e., conventional) shunt catheter. A rigid cylinder measuring 45 cm in height and 7.62 cm in diameter was perforated at intervals ranging from 0 cm (bottom of the cylinder) to 30 cm and each of the perforations was temporarily sealed with waterproof nylon tape. A threaded cap which had been perforated to accommodate insertion of a blunt 18 gauge (“18 g”) needle as well the two shunt catheters being tested was placed in the cylinder to seal one end. The shunt catheters were inserted through the perforations until the top of the catheter reached the zero perforation. The top of the blunt 18 g needle was also fixed in place at the same height. The shunt catheters were sealed into the perforations using a fast-drying liquid silicone sealant while ensuring to maintain the diameter of the shunt tubing as it passed through the perforation. A manometer was attached to the 18 g needle and placed on the side of the cylinder to allow for verification of pressure during the experimental procedure. The cylinder was filled with water to the top of the highest perforation and the shunt catheters were verified to flow. The manometer was used to confirm the pressure in cm H₂O prior to each measurement. Water was allowed to continuously flow into the top of the cylinder while measurements were being taken, thereby allowing for a constant pressure during each measurement. Each of the catheters was allowed to flow into a 100 cc graduated cylinder and a stopwatch was used to demarcate 30 second times. Starting with the highest pressure (41 cm H₂O), the volume of water over 30 seconds was measured for each catheter. At the conclusion of each time period, the next successively lower perforation was exposed to allow for measurement of flow at a slightly lower pressure and the above procedure was repeated. Pressures ranging from 0-41 cm H₂O) were evaluated.

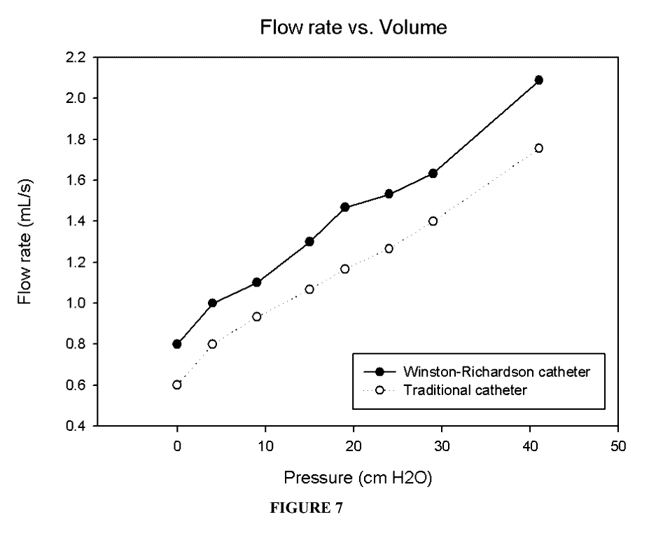

The catheter of the invention was found to flow at rates that were higher than the commercially-available catheter. See FIG. 7. The commercially-available catheter had flow rates which ranged from 0.6 mL/s at 0 cm H₂O to 1.7556 mL/s at 41 cm H₂O. The catheter of the invention had a flow rate of 0.8 mL/s at 0 cm H₂O to 2.0889 mL/s at 41 cm H₂O. Both catheters had approximately linear increases in flow rate across differing pressures as predicted by the Hagen-Poiseuille equation with all other variables being constant. Typically, the ventricular catheter of the invention provides at least about 5%, typically at least about 10%, and often at least about 15% increase in flow rate compared to a conventional ventricular catheter at 10 cm of H₂O pressure.

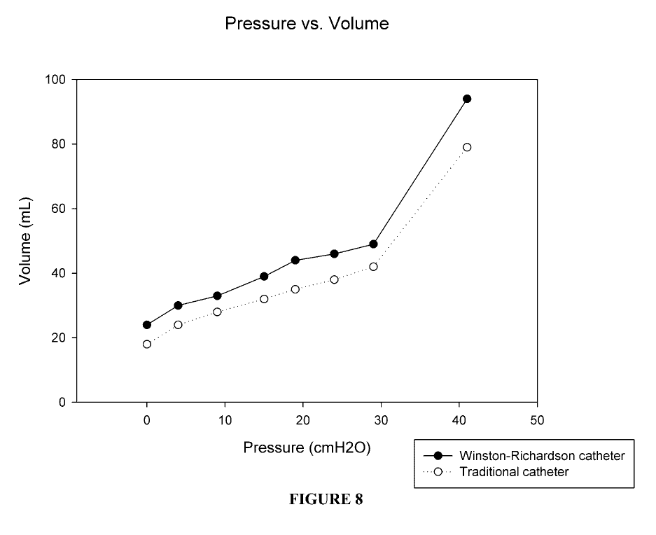

As can be seen in FIG. 8, the catheter of the invention also has a higher volume of flow compared to a conventional catheter at a given pressure. Typically, the ventricular catheter of the invention provides at least about 3%, typically at least about 5%, and often at least about 10% increase in flow volume compared to a conventional ventricular catheter at 10 cm of H₂O pressure.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A ventricular catheter comprising a tube, wherein said tube comprises:
a distal end; and
a tapered proximal end, wherein said tapered proximal end comprises a plurality of slotted openings.

2. The ventricular catheter of claim 1, wherein the interior surface of said tapered proximal end of said tube comprises a protrusion.

3. The ventricular catheter of claim 2, wherein said protrusion is located at or near the start of said tapered proximal end.

4. The ventricular catheter of claim 2, wherein said protrusion forms a ring within said interior surface.

5. The ventricular catheter of claim 1, wherein said proximal end comprises at least three slotted openings.

6. The ventricular catheter of claim 1, wherein said plurality of slotted openings runs substantially the entire length of said tapered proximal end.

7. The ventricular catheter of claim 1, wherein the length of said tapered proximal end is at least 10 mm.

8. A device comprising:
a ventricular catheter of claim 1; and
a stylet, wherein said stylet is capable of being removable fitted within said ventricular catheter, and wherein said stylet comprises:
a distal end; and
a beveled proximal end, wherein said beveled proximal end of said stylet is capable of interacting with said tapered proximal end of said ventricular catheter.

9. The device of claim 8, wherein the interior surface of said tapered proximal end of said ventricular catheter comprises a protrusion which is capable of limiting the length of said stylet inserted into said interior surface of said ventricular catheter.

10. The device of claim 9, wherein said beveled proximal end of said stylet interacts with said protrusion in said interior surface of said ventricular catheter thereby limiting the length of said stylet inserted into said ventricular catheter.

11. The device of claim 8, wherein said proximal end comprises at least three slotted openings.

12. The device of claim 8, wherein said slotted openings runs substantially the entire length of said tapered proximal end.

13. The device of claim 8, wherein the length of said tapered proximal end is at least 10 mm.

14. A method for reducing the amount and/or incident of proximal shunt catheter occlusion in a subject who is in need of a proximal shunt catheter, said method comprising placing a proximal shunt catheter of claim 1, wherein the use of said proximal shunt catheter of claim 1 reduces the amount and/or incident of proximal shunt catheter occlusion by at least 10% compared to using a conventional ventricular shunt catheter.

15. A method for reducing the amount and/or incident of ingrowth of choroid plexus in a ventricular shunt catheter, said method comprising placing a proximal shunt catheter of claim 1, wherein the use of said proximal shunt catheter of claim 1 reduces the amount and/or incident of ingrowth of choroid plexus by at least 10% compared to using a conventional ventricular shunt catheter.
